(19) United States
(12) Patent Application Publication
(10) Pub. No.: US 2012/0093743 A1
(43) Pub. Date: Apr. 19, 2012

(54) ADDITION OF FLUORIDE TO BEVERAGES

(76) Inventor: Henry M. Clayman, Miami, FL (US)

(21) Appl. No.: 12/903,380

(22) Filed: Oct. 13, 2010

(51) Int. Cl. A61K 8/21; A61Q 11/00

(52) U.S. Cl. 424/52

(57) ABSTRACT

A system and method for preventing tooth decay comprises steps or acts of adding a small dose of fluoride to a carbonated or sugar-containing beverage to prevent tooth decay by its being drunk by a user.

### FIELD OF THE INVENTION

The invention disclosed broadly relates to the field of beverages, and more particularly relates to the field of carbonated and sugar-containing beverages.

### BACKGROUND OF THE INVENTION

Fluorine is a gaseous element in the chemical halogen family. Its ionic form is called fluoride, an electro negatively charged ion, which forms stable compounds with positive ions and has a high affinity for calcium, a ubiquitous mineral in bones and teeth.

In the human oral environment fluoride in a person's saliva will absorb onto a tooth's surface and attract other minerals such as calcium, thus promoting remineralization. It also can make a tooth more decay resistant by the formation of fluorapatite, a compound formed when fluoride binds to hydroxyapatite, a complex calcium compound and a cardinal component of teeth.

Fluoride can also inhibit the formation of acids by oral bacteria such as streptococcus mutans and lactobacillus acidophilus which convert, by fermentation, dietary sugars such as glucose, sucrose and fructose into acids, e.g., lactic acid, a cause of dental demineralization and dental caries, also known as dental cavities. The salutary effects of the fluoride ion in the oral environment are the reason that various products containing fluoride are available in an effort to enhance dental health. These include mouthwashes such as ACT which contains sodium fluoride 0.05% and toothpaste, for example AQUAFRESH, incorporating sodium monofluorophosphate.

It is estimated that 70% of the water supply systems in the U.S.A. fluoridate their water prior to distribution to their customers. For example, a website of the Center for Disease Control and Prevention (CDC), National Center for Chronic Disease Prevention and Health Promotion, Division of Oral Health provides data on the fluorination of water systems, state by state. A random search showed a water system in Mississippi was fluorinated whereas a system in Idaho was not. Irrespective of the fluoride status of the water systems, many people prefer bottled water, currently devoid of fluoride, in preference to that supplied by the water system (“tap water”).

There is a huge market in the U.S.A. and other territories for carbonated beverages such as colas. A 355 ml can of Coca Cola contains 13% sugars (39 gm) and a similar can of 7-UP has 38 gm of sugars. Consumers frequently drink multiple cans of these and like beverages in a day, saturating the oral environment in the substrate for acid formation, a prime step in the inception of dental decay. It would be advantageous if a fluoride compound were added to such beverages in a modest amount in the interest of the public's dental health.

### SUMMARY OF THE INVENTION

Briefly, according to an embodiment of the invention a method includes steps or acts of adding a small dose of fluoride to a carbonated or sugar-containing beverage to prevent tooth decay.

### BRIEF DESCRIPTION OF THE SEVERAL VIEWS OF THE DRAWINGS

To describe the foregoing and other exemplary purposes, aspects, and advantages, we use the following detailed description of an exemplary embodiment of the invention with reference to the drawings, in which:

FIG. 1 is a flowchart of a method according to an embodiment of the invention.

FIG. 2 is a table on bottled water.

FIG. 3 is a map showing access to fluoridated water through the public water systems.

While the invention as claimed can be modified into alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the scope of the present invention.

### DETAILED DESCRIPTION

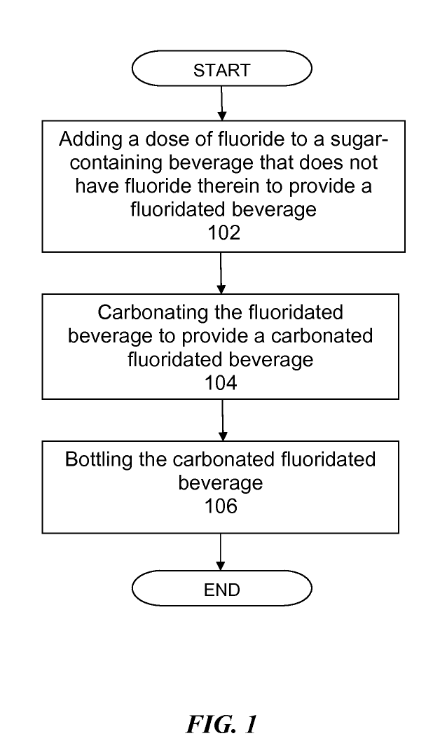

Referring to FIG. 1, we describe an invention that provides a method and a composition of matter that can be advantageously used with certain beverages for prevention of tooth decay. Soft drinks, fruit drinks, and so-called “energy” drinks are growing in popularity and generally contain a high amount of sugars such as glucose, sucrose, or fructose. Therefore, adding fluoride to these beverages to prevent tooth decay by counteracting the deleterious effects of the sugars in these beverages would be highly desirable. In step 102 a dose of fluoride is added by a bottler to a sugar-containing beverage that does not have fluoride therein to provide a fluoridated beverage. The fluoride may be added in powdered or liquid form, though liquid is preferred to avoid particulate residues in the beverage.

In step 104, the beverage is carbonated by the bottler to provide a carbonated fluoridated beverage. Lastly, in step 106, the carbonated fluoridated beverage is bottled for distribution and ultimately consumption by a user to prevent tooth decay. However, the dosage of fluoride should be in amounts small enough so that the fluoride is not detectable (by taste, color, and/or smell) to users of the beverages. Therefore, the correct dosage must be added by persons mixing and selling the beverage.

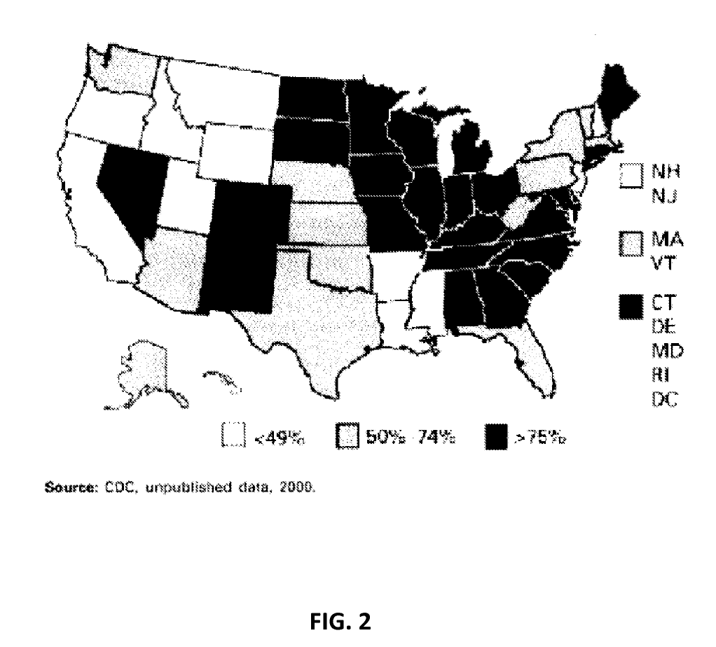

The U.S. Food and Drug Administration (FDA) has published fluoride requirements for bottled water based on the maximum daily air temperature at the point of consumption. In those locations with higher temperatures it is presumed that a consumer will drink more fluids to offset fluid loss from physiological functions such as perspiration. In the case of bottled water the FDA does not require labeling of fluoride content unless added during processing and the maximum fluoride concentration (mg/L) of permitted added fluoride is shown in FIG. 2.

The present invention addresses a problem which differs from the desirability of fluoridation to the general public. It advocates the addition of fluoride in small amounts to carbonated and other beverages which contain sugars to ameliorate the potential for dental caries caused by these sugars.

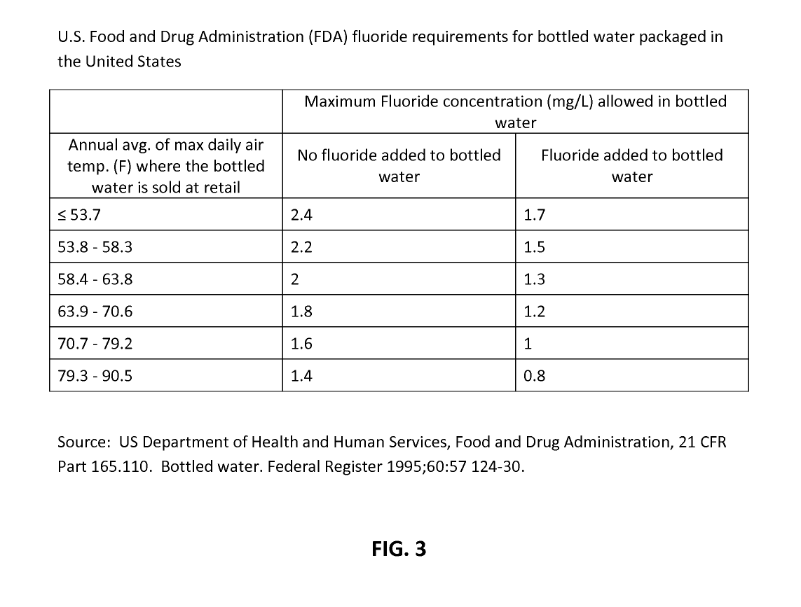

Referring to FIG. 3, there is shown a map that shows the access to fluoridated water through the public water systems in the U.S.A. (Recommendations for Using Fluoride to Prevent and Control Dental Caries in the United States. CDC MMWR Aug. 17, 2001/50 (RR14); 1-42.) “MMWR” refers to Morbidity and Mortality Report.

Considerable variation is apparent and the temperature differential across the continent is obvious. Moreover, the bottler may use local water as the substrate for the beverage and this water could be fluoridated or the water could be from an area where the public water system is devoid of fluoridation or an area with natural fluoridation. Another variable is the potential for attenuation of public or natural fluoridation by the bottler's purification system.

Given that that the sugar content of a bottler's products may vary from beverage to beverage and bottling plants tend to be local or regional, then the decision on the degree of fluoridation of a given beverage will be at the discretion of the bottler, mindful of any applicable federal regulations. The decision is based on the above factors and the premise that the added fluoridation is to offset dental caries provoked by the presence of sugars in the oral environment. The fluoridation of beverages applies whether the given beverage is in a bottle, can, carton or other container.

Therefore, while there has been described what is presently considered to be the preferred embodiment, it will understood by those skilled in the art that other modifications can be made within the spirit of the invention. The above description(s) of embodiment(s) is not intended to be exhaustive or limiting in scope. The embodiment(s), as described, were chosen in order to explain the principles of the invention, show its practical application, and enable those with ordinary skill in the art to understand how to make and use the invention. It should be understood that the invention is not limited to the embodiment(s) described above, but rather should be interpreted within the full meaning and scope of the appended claims.

We claim:

1. A method comprising:
adding a dose of fluoride to a sugar-containing beverage that does not have fluoride therein to provide a fluoridated beverage.

2. The method of claim 1 further comprising:
carbonating the fluoridated beverage to provide a carbonated fluoridated beverage.

3. The method of claim 2 further comprising:
bottling the carbonated fluoridated beverage.

4. A method for preventing tooth decay comprising:
adding a dose of fluoride to a carbonated beverage that does not have fluoride therein to provide a fluoridated beverage.

5. A composition of matter comprising:
a sugar-containing beverage; and
a dose of fluoride.

6. A composition of matter comprising:
a carbonated beverage; and
a dose of fluoride.

7. The composition of matter of claim 6 further comprising sugar.

8. The composition of matter of claim 6 further comprising a fructose fluid.

9. The composition of matter of claim 6 further comprising a glucose fluid.

10. The composition of matter of claim 6 further comprising a sucrose fluid.
